(19) United States
(12) Patent Application Publication
(10) Pub. No.: US 2019/0134227 A1
(43) Pub. Date: May 9, 2019

(54) GENERATION OF GENETICALLY ENGINEERED ANIMALS BY CRISPR/CAS9 GENOME EDITING IN SPERMATOGONIAL STEM CELLS

(71) Applicant: The Board of Regents of the University of Texas System, Austin, TX (US)

(72) Inventor: Franklin Kent HAMRA, Lewisville, TX (US)

(73) Assignee: The Board of Regents of the University of Texas System, Austin, TX (US)

(21) Appl. No.: 16/094,307

(22) PCT Filed: Apr. 5, 2017
(86) PCT No.: PCT/US2017/026070

§ 371 Date: Oct. 17, 2018

Related U.S. Application Data
(60) Provisional application No. 62/324,033, filed on Apr. 18, 2016.

(51) Int. Cl. A61K 48/00; A01K 67/027

(52) U.S. Cl. CPC A61K 48/0091; A61K 48/0066; A01K 67/0276; A01K 67/0278; A01K 2217/15; A01K 2217/072; A01K 2217/075; A01K 2217/20; A01K 2227/105

(57) ABSTRACT

The present disclosure provides methods and compositions for the production of genetically engineered animals, such as mice, by genomic editing of the spermatogonial stem cells, such as by using the CRISPR/Cas9 gene editing system. Also provided are methods of studying gene function and creating disease models.

### PRIORITY CLAIM

This application claims benefit of priority to U.S. Provisional Application Ser. No. 62/324,033, filed Apr. 18, 2016, the entire contents of which are hereby incorporated by reference.

### BACKGROUND OF THE DISCLOSURE

### 1. Field of the Disclosure

The present disclosure relates generally to the fields of molecular biology, medicine and genetics. More particularly, it concerns methods for generating genetically engineered stem cells using genome editing. Specifically, the CRISPR-Cas9 system is used to edit the genome of spermatogonial stem cells.

### 2. Description of Related Art

The ability to conditionally induce the development of stem cell lines through the process of spermatogenesis in vitro for the production of gametes would provide a long-sought-after technology for biomedical research, particularly if such protocols could be established for a variety of species. The discovery that stem cells residing within fractions of dissociated mouse and rat testis cells maintain their ability to regenerate spermatogenesis in testes of recipient mice was essential to establishing such culture systems. See Brinster et al., 1994a; Brinster et al., 1994b; Clouthier et al., 1996; Kanatsu-Shinohara et al., 2003 and Nagano et al., 1998. The ability to isolate and experimentally manipulate these stem cells has opened new doors for research on spermatozoan development, assisted reproduction, cellular therapy and genetics. See Nagano et al., 1999; Mahato et al., 2000; Mahato et al., 2001; Ogawa et al., 2000; Shinohara et al., 2006; Zhang et al., 2007; Kazuki et al., 2008; Kanatsu-Shinohara et al., 2004; Kanatsu-Shinohara et al., 2006 and Nagano et al., 2001. In view of this potential, protocols for isolating, propagating and genetically modifying fully functional rat spermatogonial stem cells in culture have been established. See Ryu et al., 2004; Hamra et al., 2004; Hamra et al., 2002; Hamra et al., 2008; Hamra et al., 2005; Ryu et al., 2005; Orwig et al., 2002 and Kanatsu-Shinohara et al., 2008. The rat was chosen as a species for these studies due to its popularity as a laboratory animal model for the study of human health and disease, and due to the lack of protocols for genetically modifying the rat germline using clonally expanded stem cells from culture. See Hamra et al., 2002. Considering the many potential applications of the laboratory rat as a research model, a cost-effective and easy-to-prepare culture medium was sought in this study for the derivation and continuous proliferation of primary rat spermatogonial stem cell lines in vitro.

In respect of this goal, media previously reported for long-term proliferation of rodent spermatogonial stem cells in vitro represent clear methodological advances for studies on the biology and applications of spermatogonia. See Kanatsu-Shinohara et al., 2003; Hamra et al., 2005; Ryu et al., 2005; Kubota et al., 2004 and Kanatsu-Shinohara et al., 2005. However, such media are relatively complex, expensive, time-consuming to prepare, plus are most effective when applied in combination with feeder layers of fibroblasts. See id. For example, the medium originally reported by Shinohara and colleagues for the successful derivation and long-term cultivation of germline stem cells from postnatal mouse testes was a pivotal breakthrough in spermatogonial research. See Kanatsu-Shinohara et al., 2003. However, Shinohara's medium is based on the proprietary, StemPro-34 medium, plus 24 individually added components, including small molecules, fetal bovine serum and a mixture of polypeptide growth factors. Serum-free derivatives of Shinohara's medium have since been formulated for spermatogonial culture, in which the serum has been replaced by the supplement, B-27. See Hamra et al., 2005 and Kanatsu-Shinohara et al., 2005. Upon inspection of components within B-27 supplement we postulated that it could be used together with key growth factors in a commonly applied nutrient mixture to formulate a more efficient spermatogonial culture medium.

Currently, specific causes of infertility in men remain a mystery in 40-60% of cases. See Bhasin et al., 1994; Sadeghi-Nejad et al., 2007; and Matzuk et al., 2008. In total, >5% of the male population is infertile, and >1% of all males are inflicted with a severe defect in sperm production termed azoospermia. See Bhasin et al., 1994; Sadeghi-Nejad et al., 2007; Barthold et al., 2003; and Bleyer, 1990. Fundamentally, because azoospermia results in an inability to reproduce by natural mating, it seems enigmatic as to why this disease remains so prevalent in the human population. Such an epidemiological trend clearly points to the existence of potent environmental factors and/or complex genetic factors that disrupt the process of sperm production (i.e. spermatogenesis) or a substantial number of de novo mutations that could arise during a lifetime to render one sterile, but otherwise healthy. See Bhasin et al., 1994; Bleyer, 1990; Reijo et al., 1995; Oates et al., 2002. In fact, this is true in numerous cases, as such de novo mutations account for several types of male-factor infertility already defined at a genetic level and increasing numbers of males are left infertile during their childhood by cancer chemotherapy. See Sadeghi-Nejad et al., 2007; Reijo et al., 1995; Bleyer et al., 1990; Oates et al., 2002; Bhasin, 2007; and Geens et al., 2008. As a new hope for many infertile men with azoospermia, a pioneering breakthrough in stem cell biology that manifested strong links between reproductive biology and genetic research was the discovery that mouse testes contained spermatogonial stem cells capable of generating fully functional sperm following isolation and transplantation into testes of another mouse. See Brinster & Zimmermann, 1994. Similar experiments soon followed in rats, and isolated mouse spermatogonia were next shown to maintain their regenerative potential after months in culture. See Clouthier et al., 1996; Nagano et al., 1998. New culture media supporting the long term proliferation of rodent spermatogonial lines in vitro have since been formulated and scientists are now on the brink of establishing conditions required to cultivate human spermatogonial lines from testis biopsies. See Kanatsu-Shinohara et al., 2003; Hamra et al., 2005; Conrad et al., 2008 and Kossack et al., 2008. Ostensibly, the ability to propagate spermatogonial lines in culture, prior to using them to produce functional spermatozoa by transplanting them back into the testes of their own donor, presents a clear strategy to cure many existing types of male infertility. Due in large part to the multipotent nature of germline stem cells however, before these breakthroughs are translated into practice it is imperative that preclinical details of such cellular therapies first be stringently evaluated in more advanced, non-human recipients of medical relevance. See Geens et al., 2008; Conrad et al., 2008; Kossack et al., 2008; Hermann et al., 2007 and Zhang et al., 2007.

In mice, embryonic stem (ES) cell-based knockout technology is very efficient for single gene targeting, and it can be combined as well with the usage of random mutagens, such as chemical mutagenic agents, viruses or transposons, for the large-scale generation of ES cell libraries, carrying different molecularly marked knockout alleles. These ES cell clones can be used for the production of knockout mice.

While this methodology is applicable for mice, it cannot be reliably employed with rats or with other laboratory animals. Furthermore, no similar or equivalent techniques to the mouse ES cell technique have yet been developed that would be applicable to a variety of animal models and not limited to one animal species like the ES cell technique in mice. For example, due to the above-mentioned technical limitation very few rat knockout strains are currently existing worldwide. This may at least partially be the result of the practicability of random mutagenesis in animals, which has proven to be questionable for several reasons. For example, the requirement of a large number of offspring, the time for rearing offspring, the costs of establishing and maintaining large-scale animal facilities are some of the factors to be considered when generating transgenic animals using random mutagenesis in animals. Accordingly, there exists a need for more advantageous methods of targeted mutagenesis in the germline that can be applied in a variety of animal models and are more practicable. Unlike technologies that micro-manipulate the early embryo, the ability to utilize donor germline stem cells for sexually transmitting experimentally introduced genomic modifications to new progeny eliminates the production of intermediate, chimeric and/or mosaic animals, and thus the time and cost associated with their production.

Current technologies used to create transgenic rats require a high level of expertise and are costly to produce. Additionally, there are many disadvantages to the currently available recipient rat models for testicular transplantation of donor stem cells. These disadvantages include but are not limited to: (1) lower germline transmission from the donor cells to progeny, due to high levels of competition from endogenous sperm cell production; (2) the need for a high number of stems cells to be transplanted into recipient testes to produce transgenic progeny; (3) a large number of progeny must be produced to yield the desired mutant rat line; and (4) the need for a high dose of cytotoxic chemicals or irradiation to achieve effective engraftment of testes by donor stem cells. In conventional protocols, moreover, the most effective levels of stem cell engraftment have not been realized because lethal doses of irradiation or cytotoxic reagents required for effective stem cell engraftment kill the recipients. Finally, production of rat lines with loss or gain of function gene mutations using female recipients requires tedious, time-consuming and prohibitively expensive micromanipulation of embryos that do not allow for the desired genomic modifications to be selected for and validated directly in an animal's germline, prior to animal production.

### SUMMARY OF THE DISCLOSURE

Thus, in accordance with the present disclosure, there is provided methods of generating a genetically engineered animal comprising contacting the spermatogonial stem cells of said animal with CRISPR guide RNA and Cas9. In particular, use of certain naturally-occurring or transgenically-generated (“genetically”) male-sterile rats as recipients for donor sperm stem cells with which the rats also are immuno-compatible. Spermatogenesis in these rats is severely disrupted, but they maintain a functional stem cell compartment. Accordingly, the transplanted sperm stem cells are free to develop into functional spermatozoa and to fertilize female rats in the absence of competition from sperm that also would be produced, were the recipients male-fertile. In this manner, 100% germline transmission of the donor cell haplotype can be achieved from a relatively low number of transplanted sperm stem cells.

Thus, pursuant to one aspect of the present disclosure, a methodology is provided for effecting germline transmission of a rat donor haplotype. The inventive method comprises the steps of (a) providing cells of a spermatogonial stem cell line that is derived from rat testes, which the cell line embodies a genetic modification generated using the CRISPR/Cas9 system, and then (b) transplanting one or more of the cells into a male-sterile recipient rat that can be, for example, the product of crossing a DAZL-deficient transgenic rat into the aforementioned genetic background, the recipient rat being immuno-compatible with the cells, such that transplanted cells develop into fertilization-competent, haploid male gametes. Also provided are media for growing spermatogonial stem cells, in addition to methods for culturing spermatogonial stem cells.

In more specific aspects, there is provided a method for generating a germline modification in the genome of a mammal comprising contacting the spermatogonial stem cells (SSCs) in said mammal with Cas9 and at least one guide RNA. The germline modification may be an insertion of a nucleic acid segment of about 10,000 to about 100,000 base pairs. The germline modification may be an insertion of a nucleic acid segment of about 10,000 to about 50,000 base pairs, about 10,000 to about 25,000 base pairs, about 10,000 to about 20,000 base pairs, or about 10,000 to about 15,000 base pairs. The germline modification may be an insertion of a nucleic acid segment of about 25 to about 200 base pairs. The germline modification may be an insertion of a nucleic acid segment of about 25 to about 150 base pairs, about 25 to about 100 base pairs, about 25 to about 75 base pairs, or about 25 to about 50 base pairs. The SSCs may be contacted with multiple guide RNAs to a plurality of target polynucleotides, such as 2, 3, 4 or 5 guide RNAs. The Cas9 may be codon optimized for expression in the SSCs.

The germline modification may comprise at least one deletion, mutation, insertion, knockout or knock-in of a gene, a gene's regulatory elements or fragment thereof. The germline modification may result in a decrease or limitation of the expression of one or more gene products. The germline modification may comprise an introduction of, or an increase in the expression of one or more gene products. The Cas9 and/or the at least one guide RNA may be provided to the SSCs through transfection or through electroporation. The method further may comprise contacting the SSCs with a single-stranded oligonucleotide. The germline modification may comprise insertion of the single-stranded oligonucleotide by non-homologous end joining (NHEJ)-mediated insertion repair or homology-directed repair (HDR).

In another embodiment, there is provide a method of treating a genetic disease in a mammal caused by a disease-causing genetic mutation comprising correcting the disease-causing mutation employing the methods set forth in paragraphs [0010] and [0011], above. The Cas9 and/or the at least one guide RNA may be provided to the SSCs through transfection or through electroporation. The Cas9 and/or the at least one guide RNA are provided to the SSCs through expression from one or more expression vectors coding thereof. The viral vector may be retroviral, lentiviral, adenoviral, adeno-associated and herpes simplex viral vectors. The expression vector may further comprise a reporter.

The at least one guide RNA and/or Cas9 may be provided to the SSCs as naked plasmid DNA, chemically-modified mRNA or protein. The mammal is a human, mouse, rat, rabbit, dog, or non-human primate. Correcting comprises insertion of a nucleic acid segment of about 10,000 to about 100,000 base pairs, or about 25 to about 200 base pairs.

In still yet another embodiment, there is provided a spermatogonial stem cell comprising a germline modification obtained according to the methods in paragraphs [0010] and [0011]. Also provided is a method of mating rats comprising mating of a male rat comprising these spermatogonial stem cell(s) with a female rat. The female rat may or may not comprise the germline modification. The germline modification may comprise insertion of a nucleic acid segment of about 10,000 to about 100,000 base pairs, or about 25 to about 200 base pairs.

In a further embodiment, there is provided a stem cell medium comprising Dulbecco's Modified Eagle Medium (DMEM), Ham's F12 nutrient mixture, from about 5-7 ng/ml glial cell-derived neurotrophic factor (GDNF), from about 5-7 ng/ml Fibroblast Growth Factor-2 (FGF2), 2-mercaptoethanol, L-glutamine, and a B27 minus vitamin A supplement solution. The ratio of Dulbecco's Modified Eagle Medium (DMEM) to Ham's F12 nutrient mixture may be about 1:1, and/or the concentration of B27 minus vitamin A supplement solution may be about 1×, and/or the concentration of 2-mercaptoethanol may be from about 50 to about 120 μM, and/or the concentration of L-glutamine may be from about 3 mM to about 10 mM, and/or the medium may further comprise G418.

Yet a further embodiment involves a stem cell composition produced by culturing a stem cell in the medium of paragraph [0015]. The stem cell may or may not be DAZ-like (DAZL) deficient. The stem cell may be cultured from about 30 days to about 150, or between about 158 and about 204 days. The stem cell may have a doubling time of between about 5 and about 9 days, or has a doubling time of no less than about 5.8, about 6, about 6.4 or about 8.4 days. The stem cell may expand no less than about 20,000 times as compared to the number of cells seeded in culture. The stem cell may be a rat spermatogonial stem cell. The rat spermatogonial stem cell may comprise one or more genetic mutations. The rat spermatogonial stem cell may further comprise a nucleotide sequence encoding one or more heterologous amino acid sequences, such as a detectable protein. The composition may be free of somatic testis cells. Correcting may comprise insertion of a nucleic acid segment of about 10,000 to about 100,000 base pairs, or insertion of a nucleic acid segment of about 25 to about 200 base pairs.

In yet a still further embodiment, there is provided a method for culturing an isolated rat spermatogonial stem cell isolated from rat testis cells comprising (a) allowing the isolated rat spermatogonial stem cell to adhere to a surface in a culture vessel; and (b) culturing the rat spermatogonial stem cell in the stem cell medium comprising Dulbecco's Modified Eagle Medium (DMEM), Ham's F12 nutrient mixture, from about 5-7 ng/ml glial cell-derived neurotrophic factor (GDNF), from about 5-7 ng/ml Fibroblast Growth Factor-2 (FGF2), 2-mercaptoethanol, L-glutamine, and a B27 minus vitamin A supplement solution. The isolated rat spermatogonial stem cell may be produced according to the method of paragraphs [0010] and [0011]. Step (b) may comprise culturing in NF9 Medium, culturing in NF9 Medium+SGF, or culturing in NF9K Medium.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-D. Rat spermatogonial tgFoxa2-FlagTag-3x knockin lines. (FIG. 1A) Oligonucleotides encoding a 3xFlag Epitope were targeted in frame into the 3′-end of Foxa2's terminal coding DNA exon sequence by homology directed repair (HDR) in a Brown Norway rat (Rattus norvegicus) spermatogonial stem cell line. (FIG. 1B) Homologous recombination of oligos into the sense (S) or antisense (A) genomic DNA strands analyzed 3 wk after co-transfection without (−G) and with (+G) pX459-T2A-Neo (co-expresses CAS9, Neomycin phosphotransferase and a gRNA targeting the 3′ end of the Foxa2 coding region), plus, ˜200 bp single stranded sense or antisense oligonucleotides (1 μl of a 100 μM/oligonucleotide stock) encoding the 3XFLAG-Tag amino acid epitope (D-Y-K-D-H-D-G-D-Y-K-D-H-D-I-D-Y-K-D) and flanked by rat Foxa2 homology arms (˜75 bp). (FIG. 1C) Right, gRNA to the Foxa2 3′ end. (M)=DNA from MEF feeder layer after harvesting spermatogonia. (BN)=DNA from BN rat. Arrowhead, expected size of knockin amplicons. Left, antibody labeling for the Flag epitope in wild-type and FOXA2 Flag rat spermatogonial lines. Scale, 40 μm. Right, western blot for FOXA2 Flag, DAZL and TUBA1a in (WT) wild-type and 3xFlag knockin (KI) rat speramtogonia. 7 out of 29 colonies (˜24%) picked were enriched for the Foxa2-3xFlag knockin (qPCR) following selection in SG Medium containing 65 μg/ml G418. (FIG. 1D) Transgene-encoded FOXA2-Flag protein was specifically immunoprecipitated from a recombinant tgFoxa2-Flag rat spermatogonial line.

FIGS. 2A-B. Clonal enrichment for inducible tgCAS9-Nickase germlines generated by HDR in donor rat spermatogonial stem cells. (FIG. 2A) CRISPR-Cas9-mediated targeted insertion of an ˜11.9 kb tgCAG-lox-Stop-lox-CAS9-Nickase-Flag-PGK-Neo transgene into the Rosa26 locus by HDR in a tgSB-T2ER-Dazl-iCre4-T2ER Sprague Dawley rat (Rattus novegicus) spermatogonial stem cell line. Approximately 15% of picked colonies (i.e., colonies #4 and #5) were enriched with the predicted targeted genomic insertion following selection in SG Medium containing 65 μg/ml G418. (FIG. 2B) Inducing tgCAS9-Nickase-Flag transmembrane functionality was validated by western blotting for the recombinant spermatogonial CAS9-NICKASE-FLAG after expanding colonies #4 and #5 into replicate, duplicate wells for treatment in culture without (−) or with (+) 100 nM 4-hydroxy-tamoxifen (4-OH-T). Western blot signals to TUBA1a provided a loading control for spermatogonial lysates. Remaining wells were expanded to 10 cm culture dishes in SG Medium and transplanted into busulfan-treated recipients.

FIGS. 3A-B. Selection for inducible tgCAS9-Nickase germlines generated by HDR in rat spermatogonial stem cells cultured on laminin in NF9 Medium+SGS. (FIG. 3A) ˜11.9 kb tgCAG-lox-Stop-lox-CAS9-Nickase-Flag-PGK-Neo transgene inserted into the genome of a wild-type Brown Norway rat (Rattus novegicus) spermatogonial stem cell line by homology directed repair. Plasmid DNA containing the tgCAG-lox-Stop-lox-CAS9-Nickase-Flag-PGK-Neo transgene targeting construct was delivered into the rat germline after sub-culturing the Brown Norway rat spermatogonial stem cell line in NF9 Medium+SGS. (FIG. 3B) PCR analysis of genomic DNA isolated from Brown Norway rat spermatogonial stem cell cultures transfected with pCAG-lox-Stop-lox-CAS9-Nickase-Flag-PGK-Neo without (Minus gRNA) or with pX458-rRosa26a (Plus gRNA). Note: PCR primers designed to detect only the correctly targeted donor genomic template specifically amplified the desired HDR targeting event (arrow), and only when spermatogonial cultures were transfected with both pCAG-lox-Stop-lox-CAS9-Nickcase-Flag-PGK-Neo and pX458-rRosa26a. Spermatogonia were thawed from passage 9 and plated on laminin ˜1.6 μg/cm² in NF9 Medium containing Spermatogonial Growth Supplement (SGS) (30% v/v). After sub-culturing for 3 passages on laminin in NF9 Medium containing SGF (NF9 Medium+SGS), spermatogonia were harvested and transfected with 10.4 μg pCAG-lox-Stop-lox-CAS9-Nickcase-Flag-PGK-Neo with or without 10.4 μg pX458-rRosa26a, as described above. Spermatogonia were then plated into 9.6 cm² wells pre-coated with ˜1.6 μg/cm² laminin matrix in NF9 Medium+SGF for ˜4 days prior to selection in NF9 Medium+SGF containing G418 (75 μg/ml) for 7 days. Spermatogonia were then cultured in NF9 Medium+SGF for 10 additional days before harvesting and analysis for targeted insertion of the pCAG-lox-Stop-lox-CAS9-Nickase-Flag-PGK-Neo transgene into the rat Rosa26 locus by HDR (FIG. 3B).

FIG. 4. Transgenic rat production. Transgenic rats were produced using heterozygous, dtTomato+ donor spermatogonia sub-cultured in NF9 Medium that were further genetically modified by knocking in a ˜11.8 kb tgCag-iCas9 Nickase (D10A) gene targeting construct. About 59% of F1 rat progeny (20 of 34 pups, n=5 litters) inherited germline tgCag-iCas9. The tgCag-iCas9 rats will facilitate gene targeting in vivo using guide RNA transgenes and viral vectors.

### DESCRIPTION OF ILLUSTRATIVE EMBODIMENTS

Here, the specific and efficient insertion of small (˜100 bp single stranded oligonucleotide knockins; in ˜25% picked colonies) and large gene targeting constructs (˜12 kb plasmid DNA construct knockin; in ˜15% of picked colonies; ˜59% of newly generated transgenic knockin rats) into the genome of rat spermatogonial stem cell lines by homologous recombination is reported. Such DNA “knockins” within Sprague Dawley and Brown Norway rat spermatogonial lines that are cultured in an improved medium including reduced GDNF and FGF2 concentrations in the medium as compared to our earlier studies.

A much more robust colonization by the donor spermatogonial lines cultured in the improved medium is also reported, and are consistently able to make genetically modified rats using individually picked rat spermatogonial lines clonally expanded/selected following gene delivery using the improved medium containing G418. Surprisingly, the stem spermatogonia operate better under all cases when the GDNF and FGF2 concentrations are lowered, and particularly, when GDNF and FGF2 are substituted with the addition of Neurturin and FGF9.

Additionally, making knockout rats by using Crispr/Cas9 to target small nucleotide mutations (Indels) into specific genes via the repair of targeted double strand DNA breaks by the Non-homologous end joining repair pathway is disclosed.

These and other aspects of the disclosure are set out in detail below.

### A. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

By “expression construct” or “expression cassette” is meant a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

A “vector” or “construct” (sometimes referred to as a gene delivery system or gene transfer “vehicle”) refers to a macromolecule or complex of molecules comprising a polynucleotide to be delivered to a host cell, either in vitro or in vivo.

A “plasmid,” a common type of a vector, is an extra-chromosomal DNA molecule separate from the chromosomal DNA that is capable of replicating independently of the chromosomal DNA. In certain cases, it is circular and double-stranded.

An “origin of replication” (“ori”) or “replication origin” is a DNA sequence, e.g., in a lymphotrophic herpes virus, that when present in a plasmid in a cell is capable of maintaining linked sequences in the plasmid and/or a site at or near where DNA synthesis initiates. As an example, an ori for EBV includes FR sequences (20 imperfect copies of a 30 bp repeat), and preferably DS sequences; however, other sites in EBV bind EBNA-1, e.g., Rep* sequences can substitute for DS as an origin of replication (Kirshmaier and Sugden, 1998). Thus, a replication origin of EBV includes FR, DS or Rep* sequences or any functionally equivalent sequences through nucleic acid modifications or synthetic combination derived therefrom. For example, the present disclosure may also use genetically engineered replication origin of EBV, such as by insertion or mutation of individual elements, as specifically described in Lindner, et. al., 2008.

A “gene,” “polynucleotide,” “coding region,” “sequence,” “segment,” “fragment,” or “transgene” that “encodes” a particular protein, is a nucleic acid molecule that is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “control elements” refers collectively to promoter regions, polyadenylation signals, transcription termination sequences, upstream regulatory domains, origins of replication, internal ribosome entry sites (IRES), enhancers, splice junctions, and the like, which collectively provide for the replication, transcription, post-transcriptional processing, and translation of a coding sequence in a recipient cell. Not all of these control elements need be present so long as the selected coding sequence is capable of being replicated, transcribed, and translated in an appropriate host cell.

The term “promoter” is used herein in its ordinary sense to refer to a nucleotide region comprising a DNA regulatory sequence, wherein the regulatory sequence is derived from a gene that is capable of binding RNA polymerase and initiating transcription of a downstream (3′ direction) coding sequence. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

By “enhancer” is meant a nucleic acid sequence that, when positioned proximate to a promoter, confers increased transcription activity relative to the transcription activity resulting from the promoter in the absence of the enhancer domain.

By “operably linked” or co-expressed” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. “Operably linked” or “co-expressed” with reference to peptide and/or polypeptide molecules means that two or more peptide and/or polypeptide molecules are connected in such a way as to yield a single polypeptide chain, i.e., a fusion polypeptide, having at least one property of each peptide and/or polypeptide component of the fusion. The fusion polypeptide is preferably chimeric, i.e., composed of heterologous molecules.

“Homology” refers to the percent of identity between two polynucleotides or two polypeptides. The correspondence between one sequence and another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions that promote the formation of stable duplexes between homologous regions, followed by digestion with single strand-specific nuclease(s), and size determination of the digested fragments. Two DNA, or two polypeptide, sequences are “substantially homologous” to each other when at least about 80%, preferably at least about 90%, and most preferably at least about 95% of the nucleotides, or amino acids, respectively match over a defined length of the molecules, as determined using the methods above.

The term “cell” is herein used in its broadest sense in the art and refers to a living body that is a structural unit of tissue of a multicellular organism, is surrounded by a membrane structure that isolates it from the outside, has the capability of self-replicating, and has genetic information and a mechanism for expressing it. Cells used herein may be naturally-occurring cells or artificially modified cells (e.g., fusion cells, genetically modified cells, etc.).

The term “stem cell” refers herein to a cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell and progenitor cell. Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPSCs”.

A “spermatogonial stem cell (SSC)” refers to a multipotent stem cell capable of self-renewal and production of daughter cells that differentiate into spermatozoa. SSCs reside within the basal layer of seminiferous tubules of the testes.

The “testes” refers to the male sex gland in the scrotum in which sperm and testosterone are produced. There is a pair of testes behind the penis in a pouch of skin called the scrotum. The testes make and store sperm, and make the male hormone testosterone.

“Spermatogenesis” refers to the process that includes all of the nuclear and cytoplasmic changes that transform the primordial germ cells of the male germ line into mature spermatocytes. The formation of mature sperm in the male testes after the onset of puberty.

“Seminiferous Tubules” are located in the testes, and are the specific location of meiosis, and the subsequent creation of gametes, namely spermatozoa. This is the tubules lining in the testes that produce sperm.

A “knockout” refers to the excision or inactivation or deletion of a gene within an intact organism or even animal model usually carried out by a method involving homologous recombination.

The term “knock-down” refers to suppression of the expression of a gene product, typically achieved by the use of antisense oligo deoxynucleotides and RNAi that specifically target the RNA product of the gene. Gene knock down refers to techniques by which the expression of one or more of an organism's genes is reduced, either through genetic modification (a change in the DNA of one of the organism's chromosomes) or by treatment with a reagent such as a short DNA or RNA oligonucleotide with a sequence complementary to either an mRNA transcript or a gene. If genetic modification of DNA is done, the result is a “knock down organism”.

A “genetically engineered” mammal, such as a mouse, is an organism or its progeny that has had its genome altered through the use of genetic engineering techniques. In some aspects, the CRISPR/Cas9 gene editing system is used to genetically modify the genome of a mouse by modification of the spermatogonial stem cells.

The term “transgenic” is defined as a genetically modified organism or its progeny that contains genetic material into which DNA from another organism has been artificially introduced. The genetic material can be in the animal's germ-cell DNA and, thus, can be transmitted from one generation to the next. In certain aspects, the term “transgenic” can include cisgenic in which the genetic material is from the same species or a species that can naturally breed with the host, xenogenic in which the genetic material can include laboratory-designed genes, linegenic in which the genetic material can be from species in the same lineage, synthetic in which the genetic material is not found in or derived from any organism, or may include genetic material which has been edited in situ, or any combination of the above.

“Electroporation” refers to the application of electric current to a living surface (as the skin or plasma membrane of a cell) in order to open pores or channels in cells or tissue through which a biological material (a drug or DNA) may pass. It is the use of electrical pulses to enable cells to take up DNA.

As used herein, the terms “disruption” or “modification” are used interchangeably herein to refer to a change in the sequence of the gene, at the DNA level. Examples include insertions, mutations, and deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination. Insertions include the insertion of entire genes, which may be of animal, plant, fungal, insect, prokaryotic, or viral origin.

In aspects of the disclosure the terms “guide RNA” refers to the polynucleotide sequence comprising the guide sequence, the tracr sequence and the tracr mate sequence. The term “guide sequence” refers to the about 20 bp sequence within the guide RNA that specifies the target site and may be used interchangeably with the terms “guide” or “spacer”. The term “tracr mate sequence” may also be used interchangeably with the term “direct repeat(s)”.

The term “Cas9” or “Cas9 nuclease” refers to an RNA-guided nuclease comprising a Cas9 protein, or a fragment thereof (e.g., a protein comprising an active or inactive DNA cleavage domain of Cas9, and/or the gRNA binding domain of Cas9).

“NF9” medium is prepared by supplementing Dulbecco's modified Eagle's medium:Ham's F12 medium 1:1 (cat. no. D8437, Sigma Inc.) with:

4 ng/ml Neurturin (cat. no. 477-MN, R&D Systems Inc.)

8 ng/ml FGF9 (cat no. 7399-F9, R&D Systems Inc.)

1× concentration B27 Supplement Minus Vitamin A (v/v) (cat. no. 12587-010, Thermo Fisher Sci. Inc.)

1× concentration antibiotic-antimycotic solution (v/v) (cat. no. 15240-062, Thermo Fisher Sci. Inc.)

4 mM L-glutamine (final concentration=6 mM) (cat no. 25030-149, Thermo Fisher Sci. Inc.)

100 μM 2-mercaptoethanol (cat. no. M3148, Sigma Inc.)

NF9+SGF medium is prepared by supplementing NF9 Medium with 10-30% (v/v) DR4 MEF Conditioned NF9 Medium. “NF9K” medium is prepared by supplementing NF9 medium with 0.5% (v/v) Knockout Serum Replacement (KSR; cat. no. 10828-010, Thermo Fisher Sci. Inc.).

### B. CRISPR/CAS9 SYSTEM

Embodiments of the present disclosure concern genome editing of SSCs to edit one or more genes in the mammalian genome using clustered regularly interspaced short palindromic repeats (CRISPR) and CRISPR-associated (Cas) proteins. See Sander and Joung, 2014.

In general, the term “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

In some embodiments, the CRISPR/Cas nuclease or CRISPR/Cas nuclease system includes a non-coding RNA molecule (guide) RNA, which sequence-specifically binds to DNA, and a Cas protein (e.g., Cas9), with nuclease functionality (e.g., two nuclease domains).

In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes.

In some embodiments, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. In some embodiments, the target site is selected based on its location immediately 5′ of a protospacer adjacent motif (PAM) sequence, such as typically NGG, or NAG. In this respect, the gRNA is targeted to the desired sequence by modifying the first 20 nucleotides of the guide RNA to correspond to the target DNA sequence.

In some embodiments, the CRISPR system induces DSBs at the target site, followed by disruptions as discussed herein. In other embodiments, Cas9 variants, deemed “nickases” are used to nick a single strand at the target site. In some aspects, paired nickases are used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

In general, a CRISPR system is characterized by elements that promote the formation of a CRISPR complex at the site of a target sequence. Typically, in the context of formation of a CRISPR complex, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The target sequence may comprise any polynucleotide, such as DNA or RNA polynucleotides. In some embodiments, the target sequence is located in the nucleus or cytoplasm of the cell. In some embodiments, the target sequence may be within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g., within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g., about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of the CRISPR complex.

As with the target sequence, in some embodiments, complete complementarity is not necessarily needed. In some embodiments, the tracr sequence has at least 50%, 60%, 70%, 80%, 90%, 95% or 99% of sequence complementarity along the length of the tracr mate sequence when optimally aligned. In some embodiments, one or more vectors driving expression of one or more elements of the CRISPR system are introduced into the cell such that expression of the elements of the CRISPR system direct formation of the CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. In some embodiments, CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of the CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to the cell.

In some embodiments, a vector comprises a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9.

In some embodiments the CRISPR enzyme is Cas9, and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. In some embodiments, one or more codons (e.g., 1, 2, 3, 4, 5, 10, 15, 20, 25, 50, or more, or all codons) in a sequence encoding the CRISPR enzyme correspond to the most frequently used codon for a particular amino acid.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of the CRISPR complex to a target sequence may be assessed by any suitable assay. For example, the components of the CRISPR system sufficient to form the CRISPR complex, including the guide sequence to be tested, may be provided to the cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of the CRISPR complex, including the guide sequence to be tested and a control guide sequence different from the test guide sequence, and comparing binding or rate of cleavage at the target sequence between the test and control guide sequence reactions.

A guide sequence may be selected to target any target sequence. In some embodiments, the target sequence is a sequence within a genome of a cell. Exemplary target sequences include those that are unique in the target genome. In some embodiments, a guide sequence is selected to reduce the degree of secondary structure within the guide sequence. Secondary structure may be determined by any suitable polynucleotide folding algorithm.

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences.

Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. In some aspects, loop forming sequences for use in hairpin structures are four nucleotides in length, and have the sequence GAAA. However, longer or shorter loop sequences may be used, as well as alternative sequences. In some embodiments, the sequences include a nucleotide triplet (for example, AAA), and an additional nucleotide (for example C or G). Examples of loop forming sequences include CAAA and AAAG. In some embodiments, the transcript or transcribed polynucleotide sequence has at least two or more hairpins. In some embodiments, the transcript has two, three, four or five hairpins. In a further embodiment, the transcript has at most five hairpins. In some embodiments, the single transcript further includes a transcription termination sequence, such as a polyT sequence, for example six T nucleotides.

In some embodiments, the CRISPR enzyme is part of a fusion protein comprising one or more heterologous protein domains (e.g., about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) betagalactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in U.S. Patent Publication 20110059502, incorporated herein by reference. In some embodiments, a tagged CRISPR enzyme is used to identify the location of a target sequence.

In some embodiments, a CRISPR enzyme in combination with (and optionally complexed with) a guide sequence is delivered to the cell. For example, CRISPR/Cas9 technology may be used to knock-down gene expression of the target antigen in the engineered cells. In an exemplary method, Cas9 nuclease (e.g., that encoded by mRNA from Staphylococcus aureus or from Streptococcus pyogenes, e.g., pCW-Cas9, Addgene #50661 (Wang et al., 2014); or nuclease or nickase lentiviral vectors available from Applied Biological Materials (ABM; Canada) as Cat. No. K002, K003, K005 or K006) and a guide RNA specific to the target antigen gene are introduced into cells, for example, using lentiviral delivery vectors or any of a number of known delivery method or vehicle for transfer to cells, such as any of a number of known methods or vehicles for delivering Cas9 molecules and guide RNAs. Non-specific or empty vector control T cells also are generated. Degree of Knockout of a gene (e.g., 24 to 72 hours after transfer) is assessed using any of a number of well-known assays for assessing gene disruption in cells.

Commercially available kits, gRNA vectors and donor vectors, for knockout of a universal tumor antigen, such any one or more of MDM2, CYP1B, HER2/neu, WT1, livin, AFP, CEA, MUC16, MUC1, PSMA, p53 or cyclin (Dl) are available, for example, from Origene (Rockville, Md.), GenScript (Atlanta, Ga.), Applied Biological Materials (ABM; Richmond, British Colombia), BioCat (Heidelberg, Germany) or others. For example, commercially available kits for knockout of hTERT via CRISPR include, for example, those available as catalog numbers K0009801, K0009802, K009803 and/or K0009804 each available from ABM. Commercially available kits for knockout of survivin via CRISPR include, for example, catalog numbers KN205935 available from Origene and catalog numbers K0184401, K0184402, K0184403, K0184404 each available from ABM. Commercially available kits for knockout of MDM2 via CRISPR include, for example, KN219518 from Origene and catalog number K1283521 from ABM. Commercially available kits for knockout of Her2/neu via CRISPR include, for example, KN212583 from Origene. Commercially available kits for knockout of Cyp 1B 1 via CRISPR include, for example, KN204074-OR available from BioCat. Commercially available kits for knockout of WT1 via CRISPR include, for example, KN220079 from Origine.

In some aspects, target polynucleotides are modified in a eukaryotic cell. In some embodiments, the method comprises allowing the CRISPR complex to bind to the target polynucleotide to effect cleavage of said target polynucleotide thereby modifying the target polynucleotide, wherein the CRISPR complex comprises the CRISPR enzyme complexed with a guide sequence hybridized to a target sequence within said target polynucleotide, wherein said guide sequence is linked to a tracr mate sequence which in turn hybridizes to a tracr sequence.

In some aspects, the methods include modifying expression of a polynucleotide in a eukaryotic cell. In some embodiments, the method comprises allowing the CRISPR complex to bind to the polynucleotide such that said binding results in increased or decreased expression of said polynucleotide; wherein the CRISPR complex comprises a CRISPR enzyme complexed with a guide sequence hybridized to a target sequence within said polynucleotide, wherein said guide sequence is linked to a tracr mate sequence which in turn hybridizes to a tracr sequence.

### C. DELIVERY OF NUCLEIC ACIDS

In some aspects, a nucleic acid encoding the guide RNA(s) and/or Cas9, is administered or introduced to the cell. The nucleic acid typically is administered in the form of an expression vector, such as a viral expression vector. In some aspects, the expression vector is a retroviral expression vector, an adenoviral expression vector, a DNA plasmid expression vector, or an AAV expression vector. In some aspects, one or more polynucleotides encoding the disruption molecule or complex, such as the DNA-targeting molecule, is delivered to the cell. In some aspects, the delivery is by delivery of one or more vectors, one or more transcripts thereof, and/or one or proteins transcribed therefrom, is delivered to the cell.

One of skill in the art would be well-equipped to construct a vector through standard recombinant techniques (see, for example, Sambrook et al., 2001 and Ausubel et al., 1996, both incorporated herein by reference). Vectors include but are not limited to, plasmids, cosmids, viruses (bacteriophage, animal viruses, and plant viruses), and artificial chromosomes (e.g., YACs), such as retroviral vectors (e.g., derived from Moloney murine leukemia virus vectors (MoMLV), MSCV, SFFV, MPSV, SNV etc), lentiviral vectors (e.g., derived from HIV-1, HIV-2, SIV, BIV, FIV, etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors.

In some embodiments, the polypeptides are synthesized in situ in the cell as a result of the introduction of polynucleotides encoding the polypeptides into the cell. In some aspects, the polypeptides could be produced outside the cell and then introduced thereto. Methods for introducing a polynucleotide construct into animal cells are known and include, as non-limiting examples stable transformation methods wherein the polynucleotide construct is integrated into the genome of the cell, transient transformation methods wherein the polynucleotide construct is not integrated into the genome of the cell, and virus mediated methods. In some embodiments, the polynucleotides may be introduced into the cell by for example, recombinant viral vectors (e.g., retroviruses, adenoviruses), liposome and the like. For example, in some aspects, transient transformation methods include microinjection, electroporation, or particle bombardment. In some embodiments, the polynucleotides may be included in vectors, more particularly plasmids or virus, in view of being expressed in the cells.

In some embodiments, delivery is via the use of RNA or DNA viral based systems for the delivery of nucleic acids. Viral vectors in some aspects may be administered directly to patients (in vivo) or they can be used to treat cells in vitro or ex vivo, and then administered to mice. Viral-based systems in some embodiments include retroviral, lentivirus, adenoviral, adeno-associated and herpes simplex virus vectors for gene transfer.

Viral vectors may be provided in certain aspects of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor-mediated endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present disclosure are described below.

Retroviruses have promise as gene delivery vectors due to their ability to integrate their genes into the host genome, transfer a large amount of foreign genetic material, infect a broad spectrum of species and cell types, and be packaged in special cell-lines (Miller, 1992).

In order to construct a retroviral vector, a nucleic acid is inserted into the viral genome in place of certain viral sequences to produce a virus that is replication-defective. In order to produce virions, a packaging cell line containing the gag, pol, and env genes—but without the LTR and packaging components—is constructed (Mann et al., 1983). When a recombinant plasmid containing a cDNA, together with the retroviral LTR and packaging sequences, is introduced into a special cell line (e.g., by calcium phosphate precipitation), the packaging sequence allows the RNA transcript of the recombinant plasmid to be packaged into viral particles, which are then secreted into the culture medium (Nicolas and Rubenstein, 1988; Temin, 1986; Mann et al., 1983). The medium containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

In some embodiments, viral and non-viral based gene transfer methods can be used to introduce nucleic acids in mammalian cells or target tissues. Such methods can be used to administer nucleic acids encoding components of a CRISPR to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, 1992; Nabel & Feigner, 1993; Mitani & Caskey, 1993; Dillon, 1993; Miller, 1992; Van Brunt, 1988; Vigne, 1995; Kremer & Perricaudet, 1995; Haddada et al., 1995 and Yu et al., 1994.

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in, e.g., U.S. Pat. Nos. 5,049,386, 4,946,787, and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91117424; WO 91116024. Delivery can be to cells (e.g., in vitro or ex vivo administration) or target tissues (e.g., in vivo administration).

In certain embodiments of the present disclosure, a nucleic acid is introduced into an organelle, a cell, a tissue or an organism via electroporation. Electroporation involves the exposure of a suspension of cells and DNA to a high-voltage electric discharge. Recipient cells can be made more susceptible to transformation by mechanical wounding. Also the amount of vectors used may vary upon the nature of the cells used, for example, about 5 to about 20 μg vector DNA per 1 to 10 million of cells may be contemplated.

In some aspects, a reporter gene which includes but is not limited to glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into the cell to encode a gene product which serves as a marker by which to measure the alteration or modification of expression of the gene product. In a further embodiment, the DNA molecule encoding the gene product may be introduced into the cell via a vector. In some embodiments, the gene product is luciferase. In a further embodiment, the expression of the gene product is decreased.

Expression cassettes included in vectors useful in the disclosure preferably contain (in a 5′-to-3′ direction) a eukaryotic transcriptional promoter operably linked to a protein-coding sequence, splice signals including intervening sequences, and a transcriptional termination/polyadenylation sequence.

1. Promoter/Enhancers

The expression constructs provided herein comprise promoter to drive expression of the programming genes. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al., 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e.g., beta actin promoter (Ng, 1989; Quitsche et al., 1989), GADPH promoter (Alexander et al., 1988, Ercolani et al., 1988), metallothionein promoter (Karin et al., 1989; Richards et al., 1984); and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007).

Tissue-specific transgene expression, especially for reporter gene expression in hematopoietic cells and precursors of hematopoietic cells derived from programming, may be desirable as a way to identify derived hematopoietic cells and precursors. To increase both specificity and activity, the use of cis-acting regulatory elements has been contemplated. For example, a hematopoietic cell-specific promoter may be used.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

Many hematopoietic cell promoter and enhancer sequences have been identified, and may be useful in methods of the disclosure. See, e.g., U.S. Pat. No. 5,556,954; U.S. Patent App. 20020055144; U.S. Patent App. 20090148425.

In particular aspects, the promoter is an inducible promoter. The activity of inducible promoters may be induced by the presence or absence of biotic or abiotic factors. Inducible promoters are a very powerful tool in genetic engineering because the expression of genes operably linked to them can be turned on or off at certain stages of development of an organism or in a particular tissue. For example, Tet-On and Tet-Off inducible gene expression systems based on the essential regulatory components of the E. coli tetracycline-resistance operon may be used. Once established in the starting cells, the inducer doxycycline (Dox, a tetracycline derivative) could control the expression system in a dose-dependent manner, allowing the precise modulation of the expression levels of programming genes. In exemplary embodiments, the inducible promoter is an rtTET-inducible Tight promoter (pTight). Thus, the pTight promoter could be used to induce expression of the multi-lineage programming genes such as ETV2, GATA2 and HOXA9 for a period of time sufficient to allow programming of the PSCs to hematopoietic precursor cells, and the expression could subsequently be turned off. The pTight promoter could also be a bi-directional promoter.

2. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the disclosure, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

Additionally, certain 2A sequence elements could be used to create linked- or co-expression of programming genes in the constructs provided in the present disclosure. For example, cleavage sequences could be used to co-express genes by linking open reading frames to form a single cistron. An exemplary cleavage sequence is the F2A (Foot-and-mouth disease virus 2A) or a “2A-like” sequence (e.g., Thosea asigna virus 2A; T2A) (Minskaia and Ryan, 2013). In particular embodiments, an F2A-cleavage peptide is used to link expression of the genes in the multi-lineage construct.

### D. GENERATION OF GENETICALLY ENGINEERED ANIMALS

In some embodiments, animals (e.g., mice) may be genetically modified using CRISPR genetic engineering tools. A genetic modification made by CRISPR/Cas9 may comprise modification or disruption of a gene. Disruption of the gene prevents expression of a functional factor encoded by the gene and comprises an insertion, deletion, or substitution of one or more bases in a sequence encoded by the gene and/or a promoter and/or an operator that is necessary for expression of the gene in the animal. The disrupted gene may be disrupted by, e.g., removal of at least a portion of the gene from a genome of the animal, alteration of the gene to prevent expression of a functional factor encoded by the gene, an interfering RNA, or expression of a dominant negative factor by an exogenous gene. Materials and methods of genetically modifying animals are further detailed in U.S. Pat. No. 8,518,701; U.S. 2010/0251395; and U.S. 2012/0222143, which are hereby incorporated herein by reference

In some embodiments, the modification results in reduced expression of the target polynucleotide sequence(s). The terms “decrease,” “reduced,” “reduction,” and “decrease” are all used herein generally to mean a decrease by a statistically significant amount. The decrease may be a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e., absent level as compared to a reference sample), or any decrease between 10-100% as compared to a reference level.

In some embodiment, the modification results in increased expression of the target polynucleotide sequence(s). The terms “increased”, “increase” or “enhance” or “activate” are all used herein to generally mean an increase by a statically significant amount, such as an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

In some embodiments, the genetic modification results in correction of the target polynucleotide sequence from an undesired sequence to a desired sequence. The CRISPR/Cas9 system can be used to correct any type of mutation or error in a target polynucleotide sequence. For example, the CRISPR/Cas9 system can be used to insert a nucleotide sequence that is missing from a target polynucleotide sequence due to a deletion. The CRISPR/Cas9 system can also be used to delete or excise a nucleotide sequence from a target polynucleotide sequence due to an insertion mutation. In some instances, the CRISPR/Cas9 system can be used to replace an incorrect nucleotide sequence with a correct nucleotide sequence (e.g., to restore function to a target polynucleotide sequence that is impaired due to a loss of function mutation, i.e., a SNP).

1. Rat Spermatogonial Stem Cells

Rat spermatogonial stem cells are rat cell types that when transplanted into the testes can develop into fertilization competent haploid gametes, such as spermatids or spermatozoa. Natural mating or breeding of the recipient with a female rat bypasses any requirement for assisted fertilization methods. Additionally, mating can include methods such as but not limited to in vitro fertilization, intrauterine transfer, or oocyte injection with nuclei. Sperm produced from the spermatogonial lines in the recipient rats are highly effective at transmitting the donor haplotype to F1 and F2 progeny by mating, thereby rendering unnecessary conventional embryo manipulations to generate rat progeny from donor spermatogonial lines.

The combination of (i) rat spermatogonial lines that can be propagated in culture and (ii) male-sterile recipient rats is an optimal vector for germline transmission of natural or genetically modified rat genomes. This approach will allow for preservation of existing rat lines and the production of new transgenic rat lines important for science and industry. For example, the invention offers avenues to the establishment of new transgenic mutant rat models, for the study of human biology and disease.

Spermatogonial stem cell lines are derived from testes of rats, such as inbred Fischer 344 rats, that embody a desirable genetic background. The derived lines are expanded in cell number over subsequent subculturing steps in vitro, preferably using SG medium, which is described below. Furthermore, DAZL-deficient transgenic rats are obtained from a cross into the desired rat genetic (e.g., Fischer 344) background. The male-sterile recipient animals thus produced are immuno-compatible with the derived spermatogonial lines.

The phrase “spermatogonial stem cells” in this description denotes stem cells isolated from the testis. Spermatogonial stem cells are incapable of fertilizing an egg cell but can give rise to cells that develop into sperm and that produce viable offspring. Isolated spermatogonial stem cells can be cultured for a prolonged time period without losing their properties and can efficiently repopulate the testes of suitable recipient male animals described, for instance, in Oatley et al., 2006.

Transplanting cells of a spermatogonial line, as described above, into an immuno-compatible, male-sterile recipient allows the cells to develop into fertilization-competent, haploid male gametes. Since there is negligible endogenous sperm competition generated by the testes of the male-sterile recipient's testes, the inventive approach effects maximal germline transmission of donor haplotypes from the transplanted spermatogonial cells.

A pure genetic background is more desirable than a mixed genetic background for testing the effects of gene mutations and transgenes in animal studies. Moreover, conventional production of rat lines with loss- or gain-of-function mutations requires tedious, time-consuming, and prohibitively expensive micromanipulation of embryos. The disclosed methods also can use spermatogonia from inbred rats to bypass any requirement for in vitro manipulating of early rat embryos of inbred strains. This reduces the time, cost, and effort required to produce mutations in inbred or outbred rat lines.

The phrase “selectable marker” is employed here to denote a protein that enables the separation of cells expressing the marker from those that lack or do not express it. The selectable marker may be a fluorescent marker, for instance.

Expression of the marker by cells having successfully integrated the transposon allows the isolation of these cells using methods such as, for example, FACS (fluorescent activated cell sorting). Alternatively, expression of a selectable marker may confer an advantageous property to the cell that allows survival of only those cells carrying the gene.

For example, the marker protein may allow for the selection of the cell by conferring an antibiotic resistance to the cell. Consequently, when cells are cultured in medium containing said antibiotic, only cell clones expressing the marker protein that mediates antibiotic resistance are capable of propagating. By way of illustration, a suitable marker protein may confer resistance to antibiotics such as ampicillin, kanamycin, chloramphenicol, tetracycline, hygromycin, neomycin or methotrexate. Further examples of antibiotics are penicillins: ampicillin HCl, ampicillin Na, amoxycillin Na, carbenicillin disodium, penicillin G, cephalosporins, cefotaxim Na, cefalexin HCl, vancomycin, cycloserine. Other examples include bacteriostatic inhibitors such as: chloramphenicol, erythromycin, lincomycin, spectinomycin sulfate, clindamycin HCl, chlortetracycline HCl. Additional examples are marker proteins that allow selection with bactericidal inhibitors such as those affecting protein synthesis irreversibly causing cell death, for example aminoglycosides such as gentamycin, hygromycin B, kanamycin, neomycin, streptomycin, G418, tobramycin. Aminoglycosides can be inactivated by enzymes such as NPT II which phosphorylates 3′-OH present on kanamycin, thus inactivating this antibiotic. Some aminoglycoside modifying enzymes acetylate the compounds and block their entry in to the cell. Marker proteins that allow selection with nucleic acid metabolism inhibitors like rifampicin, mitomycin C, nalidixic acid, doxorubicin HCl, 5-fluorouracil, 6-mercaptopurine, antimetabolites, miconazole, trimethoprim, methotrexate, metronidazole, sulfamethoxazole are also examples for selectable markers.

The term “rat” refers to a member of the genus Rattus, such as the black rat, Rattus rattus, and the brown rat, Rattus norvegicus. The laboratory rat is one of the most extensively studied model organisms for human disease and, hence, is the major animal model in the initial stages of drug development. In contrast to mice, a limitation of the rat model has been the lack of technology for generating “defined” genetic mutants. Such defined genetic mutants, where precise changes to a gene sequence or function are made without perturbing the rest of the genome, are critical for determining gene function with a high degree of certainty and for creating reliable genetic models for human disease. Due to the lack of technology for generating defined mutants in rats, genetic manipulation approaches were not successful in the rat genome so far and genome research in the rat to connect genetic backgrounds with certain phenotypes is lagging behind.

Once generated, a spermatogonial cell line is transplanted into a male-sterile recipient rat. In a preferred embodiment of the invention, spermatogonial cell lines are transplanted into transgenically created male-sterile recipients. Alternatively, spermatogonial cell lines are transplanted into recipients that have naturally occurring genetic mutations that cause male-sterility.

Examples of naturally occurring mutations that generate male sterile rats include but are not limited to: rats that have mutations in FKBP6; rats that have altered function of the pituitary-gonadal axis, see Kamtchouing et al., 1991, for example; and rats that have a mutant BIL/1. Illustrative of transgenically generated male sterile rat are the DAZL-deficient transgenic rat and rats that express the HSV type 1 thymidine kinase protein.

The term “about” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods and maintain the viability of the spermatogonial stem cell line.

2. Editing Spermatogonial Stem Cells

Embodiments of the present disclosure concern genome editing of spermatogonial stem cells which are responsible for the production of spermatozoa by spermatogenesis (Naito et al., 1998) and hence an appropriate target for germline modification. Similar to other tissue-specific stem cells, SSCs are rare, representing only 0.03 percent of all germ cells in rodent testes (Tegelenbosch & de Rooij 1993). This is because SSCs are heavily outnumbered by the differentiating spermatogonia, spermatocytes, spermatids and sperm that they produce. SSCs are defined like all other stem cells, by their ability to balance self-renewing divisions and differentiating divisions. This balance maintains the stem cell pool and meets the proliferative demand of the testis to produce millions of sperm each day. Studies of SSCs are complicated because these cells are few in number and no unique identifying characteristics have been reported to date (Phillips et al., 2010); incorporated herein by reference).

SSCs arise from gonocytes in the postnatal testis, which arise from primordial germ cells (PGCs) during foetal development. PGCs are a transient cell population that is first observed as a small cluster of alkaline phosphatase-positive cells in the epiblast stage embryo at about 7-7.25 days post coitum (dpc). PGC specification is dependent on the expression of BMP4 and BMP8b from the extraembryonic ectoderm (Ginsburg et al., 1990).

Previous studies have generated transgenic mice by spermatogonial stem cell manipulation in vitro using either recombinant retroviruses or lentiviruses to infect spermatogonial stem cells in vitro and then transplant the cells into the testes of isogenic adult male mice, however, in some cases the recipient mice were unreceptive to the donor spermatogonial cells.

Accordingly, embodiments of the present disclosure concern genetic modification of SSCs by direct in vivo manipulation. In some aspects, the SSCs are contacted in vivo with the guide RNA(s) and/or Cas9 by direct injection in the testis. For example, lentivirus expressing the guide RNA(s) and/or Cas9 may be injected into the testis of the mouse (Sehgal et al., 2014).

Additionally, transgenic mice have shown to be generated by electroporation of an expression construct into the testes of adult male mice as described in U.S. Pat. No. 8,373,018, incorporated herein by reference. Briefly, a recombinant lentivirus is injected into the space between the seminiferous tubules of a testis of a male mouse followed by application of an electric current, thereby producing spermatozoa expressing the genetic modification within a span of about 30-35 days. Thus, methods of the present disclosure can involve electroporation of the testis for introduction of the nucleic acid.

Thus, certain embodiments of the present disclosure concern methods for the injection of Cas9 mRNA and guide-RNA(s), such as lentivirus with expression thereof, into the testis of a mouse followed by electroporation. Recombinant lentiviruses expressing Cas9 and/or guide-RNA(s) are injected into the intertubular spaces of the testis targeting undifferentiated spermatogonia present in the seminiferous tubules. The intertubular spaces allows the lentivirus to infect undifferentiated spermatogonial cells located at the basement of the seminiferous tubules. The undifferentiated spermatogonial cells in mice produce mature spermatozoa in about 30 to about 35 days. Thus, the mice transduced by lentivirus will have mature sperm with modified allele(s) at about 30-35 days. These mice can then be mated with wild-type or Cas9 transgenic female mice. Standard breeding techniques can be used to create animals that are homozygous for the modified allele(s).

In certain embodiments of the present disclosure, a genetically engineered mammal (e.g., a mouse) is generated by CRISPR-mediated genome editing comprising the steps of constructing a lentivirus expressing the guide-RNA(s) and/or Cas9, transduction and/or electroporation of the lentivirus into the testis of the male mouse, and mating of the transduced mouse with a wild-type female to obtain progeny with the modified allele(s). In some aspects, the guide-RNA(s) and/or Cas9 are injected into the efferent duct followed by electroporation of the testes for in vivo transfection (Michealis et al., 2014); incorporated herein by reference).

In certain aspects, the present disclosure involves genetic modification of SSCs to inactivate a gene or a plurality of genes by multiplex genomic editing using the CRISPR/Cas9 system described herein. Thus, embodiment include targeting 1, 2, 3, 4, 5 or more targets in a multiplex approach. Some traits, like cancer, are caused on the basis of mutations at multiple genes (see APC/p53). In addition numerous disease traits are so-called Complex traits that manifest as a result of the influence of alleles at more than one gene. For example, diabetes, metabolism, heart disease, and neurological diseases are considered complex traits. Thus, embodiments include animal models that are heterozygous and homozygous for individual alleles, or in combination with alleles at other genes, in different combinations. For example mature onset diabetes of the young (MODY) loci cause diabetes individually and additively, including; MODY 1 (HNF4a), MODY 2 (GCK), MODY 3 (HNF1a), MODY 4 (Pdx1), MODY 5 (HNF-Iβ), MODY 6 (eurogenic differentiation 1), MODY 7 (KLF11), MODY 8 (CEL), MODY 9 (PAX4), MODY 10 (INS), MODY 11 (BLK). Livestock cells or embryos can be subjected to multiplex editing of numerous genes for animal modelling, including various disease modeling targets: APC, ApoE, DMD, GHRHR, HR, HSD11B2, LDLR, NF1, NPPA, NR3C2, p53, PKD1, Rbm20, SCNN1G, tP53, DAZL, FAH, HBB, IL2RG, PDX1, PITX3, Runxl, RAG2, GGTA.

In further embodiments, genome-wide loss-of-function mutations and novel phenotypes can be screened by generate lentivirus using a pooled CRISPR library (such as GeCKO V2).

### E. USE OF METHODS FOR GERMLINE MODIFICATIONS

The genetically modified animals of the present disclosure and progeny thereof as well as genetically modified SSCs, other cells, and sperm are useful in studying the mechanisms behind gene function such as by loss-of-function modifications as well as creating disease models. Additionally, the animals can be used to test compounds useful in treating and diagnosing human diseases. Accordingly, methods of screening assays are provided herein for identifying modulators, i.e., candidate or test compounds or agents (e.g., peptides, cyclic peptides, peptidomimetics, small molecules, small organic molecules, or other drugs) which effect (i.e., modulate, inhibit, reduce, prevent or reverse) diseases.

For example, the genetically modified animals provided herein can be used as models for cancer, autoimmune diseases or genetic disorders. In some embodiments, the disorder is a monogenic disorder. In some embodiments, the disorder is a multigenic disorder. In some embodiments, the disorder is a disorder associated with one or more SNPs. Exemplary disorders associated with one or more SNPs include a complex disease described in U.S. Pat. No. 7,627,436, Alzheimer's disease as described in PCT International Application Publication No. WO/2009/112882, inflammatory diseases as described in U.S. Patent Application Publication No. 2011/0039918, polycystic ovary syndrome as described in U.S. Patent Application Publication No. 2012/0309642, cardiovascular disease as described in U.S. Pat. No. 7,732,139, Huntington's disease as described in U.S. Patent Application Publication No. 2012/0136039, thromboembolic disease as described in European Patent Application Publication No. EP2535424, neurovascular diseases as described in PCT International Application Publication No. WO/2012/001613, psychosis as described in U.S. Patent Application Publication No. 2010/0292211, multiple sclerosis as described in U.S. Patent Application Publication No. 2011/0319288, schizophrenia, schizoaffective disorder, and bipolar disorder as described in PCT International Application Publication No. WO/2006/023719A2, bipolar disorder and other ailments as described in U.S. Patent Application Publication No. U.S. 2011/0104674, colorectal cancer as described in PCT International Application Publication No. WO/2006/104370A1, a disorder associated with a SNP adjacent to the AKT1 gene locus as described in U.S. Patent Application Publication No. U.S. 2006/0204969, an eating disorder as described in PCT International Application Publication No. WO/2003/012143A1, autoimmune disease as described in U.S. Patent Application Publication No. U.S. 2007/0269827, fibrostenosing disease in patients with Crohn's disease as described in U.S. Pat. No. 7,790,370, and Parkinson's disease as described in U.S. Pat. No. 8,187,811, each of which is incorporated herein by reference in its entirety.

Animals of any species, including, but not limited to, mice, rats, rabbits, guinea pigs, pigs, micro-pigs, goats, and non-human primates, e.g., baboons, monkeys, and chimpanzees may be used to generate disease animal models. These systems may be used in a variety of applications. Such assays may be utilized as part of screening strategies designed to identify agents, such as compounds that are capable of ameliorating disease symptoms. Thus, the animal- and cell-based models may be used to identify drugs, pharmaceuticals, therapies and interventions that may be effective in treating disease.

Additionally, the CRIPSR-based gene correction of the present disclosure has many clinical and preclinical (e.g., research) applications. For example, CRISPR-based gene correction can be used to correct genes in which mutations lead to disease. For example, any disease characterized by small base alterations including insertions and deletions such as, but not restricted to, epidermolysis bullosa, osteogenesis imperfecta, dyskeratosis congenital, the mucopolysaccharidoses, muscular dystrophy, cystic fibrosis (CFTR), fanconi anemia, the sphingolipidoses, the lipofuscinoses, adrenoleukodystrophy, severe combined immunodeficiency, sickle-cell anemia, thalassemia, and the like.

### F. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Example 2—Precise Genomic Modifications in Rat Germlines by Homology Directed Repair in Spermatogonial Stem Cells

Rat Spermatogonial Stem Cell Lines. Rat (Rattus norvegicus) spermatogonial lines were derived from laminin-binding spermatogonia (Hamra, 2002) isolated from individual homozygous SD-Tg(SB-T2ER-Dazl-iCre4-T2ER)4Fkh Sprague Dawley rats, heterozygous SD-Tg(ROSA-EGFP)2-4Reh Sprague Dawley rats, wildtype Brown Norway rats, or transgenic BN-Tg(Dazl-dtTomato)Fkh Brown Norway rats. SD-Tg(SB-T2ER-Dazl-iCre4-T2ER)4Fkh rats are referred to as tgGCS-iCre rats because they exhibit germ cell specific expression of a 4-hydroxytamoxifen inducible form of T2ER-CRE-T2ER recombinase (iCRE) (unpublished FKH). SD-Tg(ROSA-EGFP)2-4Reh rats are referred to as tgGCS-EGFP rats because they exhibit germ cell specific expression of enhanced green fluorescent protein (EGFP) (Cronkhite et al., 2005). Inbred Brown Norway rats were from original wildtype stock (Charles Rivers, Inc.). Spermatogonial lines were derived and propagated on feeder layers of irradiated DR4 mouse embryonic fibroblasts (DR4 MEFs) as previously detailed (Chapman et al., 2011; Wu et al., 2009), but using Spermatogonial Culture (SG) Medium containing 6 ng/ml bFGF (PGF0023, Life Technologies, Inc.) and 6 ng/ml GDNF (512-GF, R&D Systems, Inc.), or, NF9 Medium containing 4 ng/ml Neurturin and 8 ng/ml FGF9.

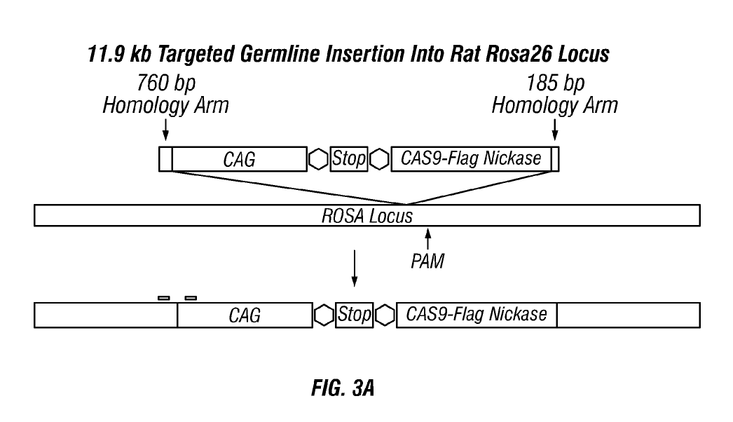

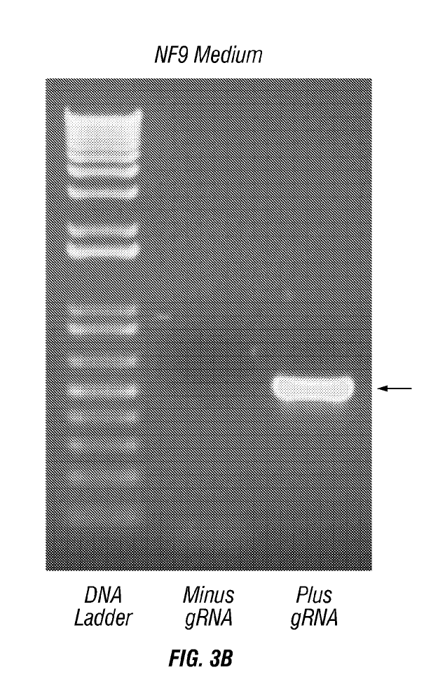

In some experiments (FIGS. 3A-B), gene targeting was conducted in Brown Norway rat spermatogonial stem cells derived and cultured in NF9 Medium containing: Hyclone Ham's F12 medium 1:1 (cat. no. SH30023.02, Thermo Fisher Sci., Inc.), 4 ng/ml Neurturin (cat. no. 477-MN, R&D Systems, Inc.), 8 ng/ml FGF9 (cat. no. 7399-F9, R&D Systems, Inc.), 1× concentration of B27 Supplement Minus Vitamin A (v/v) (cat. no. 12587-010, Thermo Fisher Sci., Inc.), 1× concentration antibiotic-antimycotic solution (v/v) (cat. no. 15240-062, Thermo Fisher Sci., Inc.), 4 mM L-glutamine (final concentration=6.5 mM) (cat. no. 25030-149, Thermo Fisher Sci., Inc.) and 100 μM 2-mercaptoethanol (cat. no. M3148, Sigma, Inc.), 30% v/v Spermatogonial Growth Supplement (SGS).

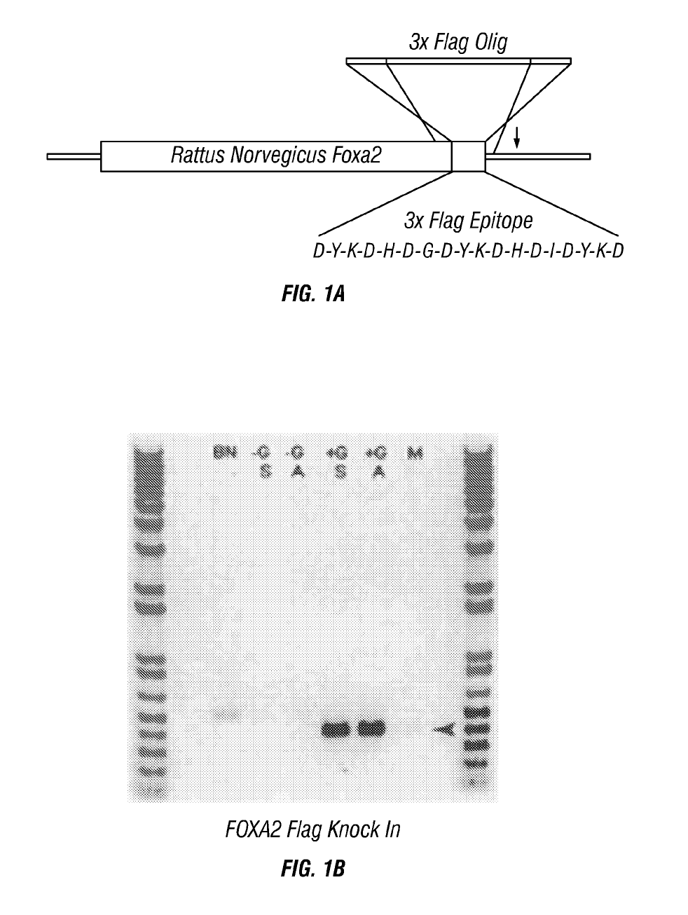

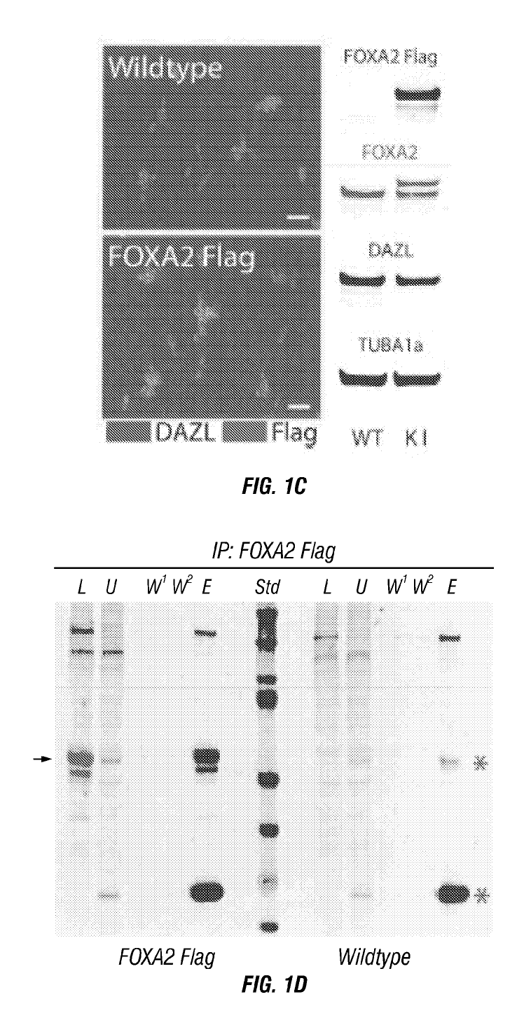

Targeted Genomic Insertion of Single Stranded Oligonucleotide Constructs into Rat Spermatogonial Lines by HDR (FIGS. 1A-D).

To generate FOXA2-3xFLAG tagged rat spermatogonia, a Brown Norway rat (Rattus norvegicus) spermatogonial stem cell line was harvested at passage 4 and co-transfected in suspension with 5.25 μg pX459-T2A-Neo (co-expresses CAS9, Neomycin phosphotransferase and a gRNA targeting the 3′ end of the Foxa2 coding region), plus, ˜200 bp single stranded sense or antisense oligonucleotides (1 μl of a 100 μM/oligonucleotide stock) encoding the 3XFLAG-Tag amino acid epitope (D-Y-K-D-H-D-G-D-Y-K-D-H-D-I-D-Y-K-D) and flanked by rat Foxa2 homology arms (˜75 bp) using the Neon Transfection System as described above. Oligonucleotides encoding a 3xFlag Epitope were targeted in frame into the 3′-end of rat spermatogonial Foxa2's terminal coding DNA exon sequence by homology directed repair (HDR).

To prepare the transfection suspension, the spermatogonia (3.1×10⁶) were harvested from a 10 cm dish at passage 4 by pipetting to rinse spermatogonia off the attached feeder layer of DR4 MEFs, and then cultured on a single 10 cm gelatin-coated culture dish under standard conditions (SG Medium at 36.5° C., 5% CO₂; SG Medium) for ˜3 hr to deplete any contaminating DR4-MEFs, and to enrich for stem spermatogonia that selectively survived the passaging procedure. Suspensions of the gelatin-non-binding stem spermatogonia were then harvested from the gelatin coated dish, washed 1× in 4 ml PBS, and then the pellet was suspended to ˜2.1×10⁶ spermatogonia with 400 μl Neon Resuspension buffer R, and then co-transfected in suspension after adding the DNA plasmid-oligonucleotide mixture (made up to 10 μl in TE buffer).

Spermatogonia electroporated in each tip were added into a single 15 ml aliquot of SG Medium that was divided into 4, 9.6 cm² wells of a 6-well culture dish (˜3.75 ml/well) containing irradiated DR4 MEFs, at a pre-transfection equivalent of 0.525×10⁵ transfected spermatogonia/well (Passage 5), ˜2 days prior to starting selection in SG Medium containing 65 μg/ml G418 for 8 days, prior to spiking cultures with fresh irradiated DR MEFs in SG Medium without G418. Spermatogonial cultures were maintained under standard conditions without G418 for 3 additional days prior to passaging cells from each 9.6 cm² well into 2, 3.8 cm² wells (˜15,000 spermatogonia/well) containing fresh irradiated DR MEFs in SG Medium (Passage 6). Cultures were maintained for 13 days under standard conditions prior to passaging cells into 3, 9.6 cm² wells at 10,000 to 30,000 spermatogonia/well (for picking clonally expanded colonies) containing fresh irradiated DR MEFs in SG Medium (Passage 7). 21 days after passaging, individual colonies were picked into 48-well plates (0.96 cm²; fresh irradiated DR4 MEFs were spiked into cultures at ˜3.5×10⁴/well at day 14 following passaging). Picked colonies were each sub-cultured to ˜10⁶ spermatogonia under standard conditions for western blotting and immunoprecipitation experiments.

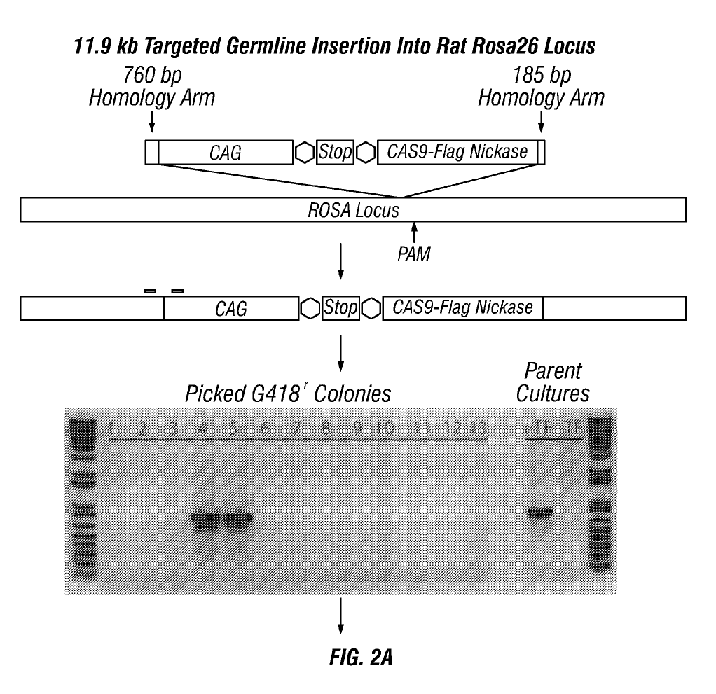

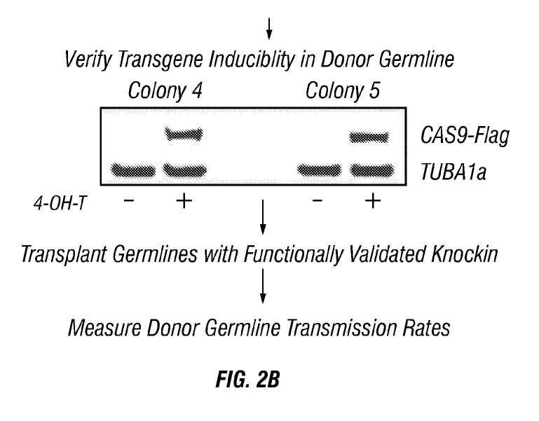

Targeted Genomic Insertion of Large Plasmid DNA Constructs into Rat Spermatogonial Lines by HDR (FIGS. 2A-B).

To generate novel strains of inducible CAS9-Nickase transgenic rats (tgGCS-CAS9-Nickase), a tgGCS-iCre rat spermatogonial line with precisely targeted genomic modifications were made by experimentally inserting the donor tgCAG-lox-Stop-lox-CAS9-Nickase-Flag-PGK-Neo transgene into the rat Rosa26 genomic locus of tgGCS-iCre rats by homology directed repair (HDR). As donor construct substrate for HDR into the rat spermatogonial genome, the tgCAG-lox-Stop-lox-CAS9-Nickase-Flag-PGK-Neo transgene was flanked by 760 bp and 185 bp rat Rosa26 homologous nucleotide “arms” (FIG. 2A).

The donor pCAG-lox-Stop-lox-CAS9-Nickase-Flag-PGK-Neo transgene-containing DNA plasmid was delivered into the rat germline by Neon transfection of the Sprague Dawley tgGCS-iCRE spermatogonial stem cell line. Spermatogonia (3.9×10⁶) were harvested from 2×10 cm dishes at passage 8 by pipetting to rinse spermatogonia off the attached feeder layer of DR4 MEFs, and then cultured on a single 10 cm gelatin-coated culture dish under standard conditions (SG Medium at 36.5° C., 5% CO₂; SG Medium changed every ˜49 hr) for 3 hr to deplete any contaminating DR4-MEFs, and to enrich for stem spermatogonia that selectively survived the passaging procedure. Suspensions of the gelatin-non-binding stem spermatogonia were then harvested from the gelatin coated dish (yield=˜2.2×10⁶), washed 1× in 4 ml PBS, and then concentrated to ˜2.1×10⁶ cells by suspending the pellet with 400 μl Neon Resuspension buffer R, and then co-transfected in suspension after adding 25 μl of a DNA plasmid mixture containing 10.4 μg pCAG-lox-Stop-lox-CAS9-Nickase-Flag-PGK-Neo and 10.4 μg pX458-rRosa26a (gRNA containing plasmid) made up in TE Buffer. For co-transfection, the Neon Transfection System was used to deliver 2 pulses at 1100 V, 20 ms to electroporate the spermatogonia-DNA plasmid suspension by dividing it into 4 equal 100 μl suspensions using Neon 100 μl tips (MPK10025) that were inserted into the pipet chamber containing 3 ml Neon Electrolyte Buffer E2. Spermatogonia electroporated in each tip were added into a single 16 ml aliquot of SG Medium that was divided into 6, 9.6 cm² wells of a 6-well culture dish (˜2.67 ml/well) containing irradiated DR4 MEFs, at a pre-transfection equivalent of ˜3.5×10⁵ transfected spermatogonia/well.

Transfected spermatogonia were maintained under standard conditions on irradiated DR4 MEFs in SG Medium for two weeks prior to their initial passage post-transfection (Passage 9: ˜1.3×10⁶ yield). Harvested cells were plated at ˜7×10⁴ cells/well into 12, 9.6 cm² wells on 2, 6-well plates for ˜2 days prior to starting selection in SG Medium containing 65 μg/ml G418 for 6 days. Spermatogonial cultures were maintained under standard conditions without G418 for 2 additional days prior to passaging (Passage 11) onto 2×10 cm gelatin-coated dishes for ˜3 hr to deplete contaminating DR4 MEFs, and to enrich for surviving stem spermatogonia. The unbound spermatogonial suspension was harvested from the gelatin-coated plates and plated onto fresh irradiated DR4 MEFs at ˜2.5×10⁴ spermatogonia/well for clonal expansion in 4, 9.6 cm² wells for 28 days prior to picking individual colonies into 48-well plates (0.96 cm²; fresh irradiated DR4 MEFs were spiked into cultures at ˜3.5×10⁴/well at day 14 following passaging). Picked colonies were each sub-cultured to ˜10⁶ spermatogonia under standard conditions prior to transplantation at ˜2.5×10⁵ cells/testis/colony into busulfan-treated wild-type rats (Hamra, 2002).

FIGS. 3A-B. Selection for Inducible tgCAS9-Nickase Germlines Generated by HDR in Rat Spermatogonial Stem Cells Cultured on Laminin in NF9 Medium+SGS.

In some experiments, the donor pCAG-lox-Stop-lox-CAS9-Nickase-Flag-PGK-Neo transgene-containing DNA plasmid (FIG. 3A) was delivered into the rat germline by Neon transfection of a Brown Norway rat spermatogonial stem cell line cultured in NF9 Medium. Spermatogonial were thawed from passage 9 and plated on laminin ˜1.6 μg/cm² in NF9 Medium containing Spermatogonial Growth Supplement (SGS) (30% v/v). After sub-culturing for 3 passages on laminin in NF9 Medium containing SGF (NF9 Medium+SGS), spermatogonia were harvested and transfected with 10.4 μg pCAG-lox-Stop-lox-CAS9-Nickcase-Flag-PGK-Neo with or without 10.4 μg pX458-rRosa26a, as described above. Spermatogonia were then plated into 9.6 cm² wells pre-coated with ˜1.6 μg/cm² laminin matrix in NF9 Medium+SGF for ˜4 days prior to selection in NF9 Medium+SGF containing G418 (75 μg/ml) for 7 days. Spermatogonia were then cultured in NF9 Medium+SGF for 10 additional days before harvesting and analysis for targeted insertion of the pCAG-lox-Stop-lox-CAS9-Nickase-Flag-PGK-Neo transgene into the rat Rosa26 locus by HDR (FIG. 3B).

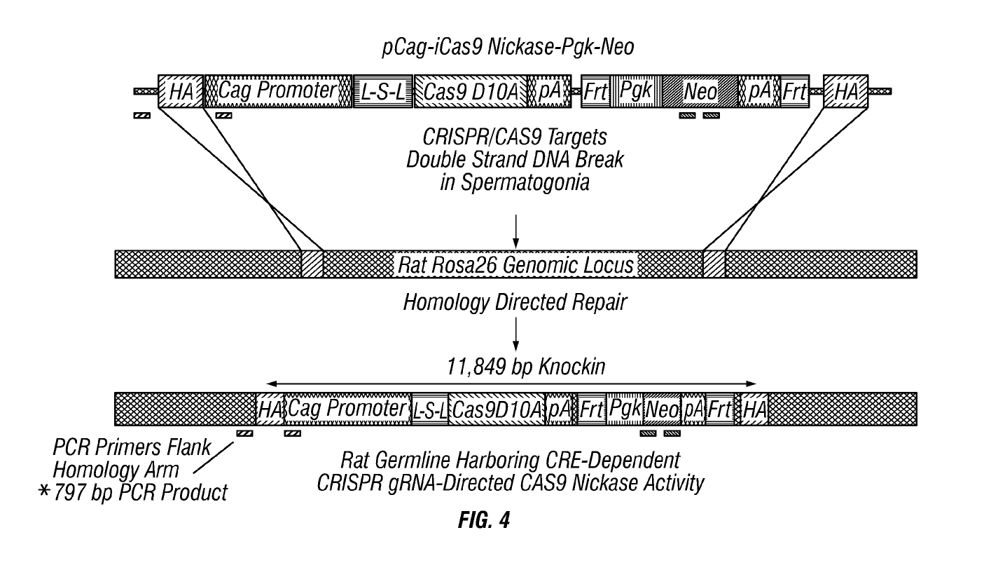

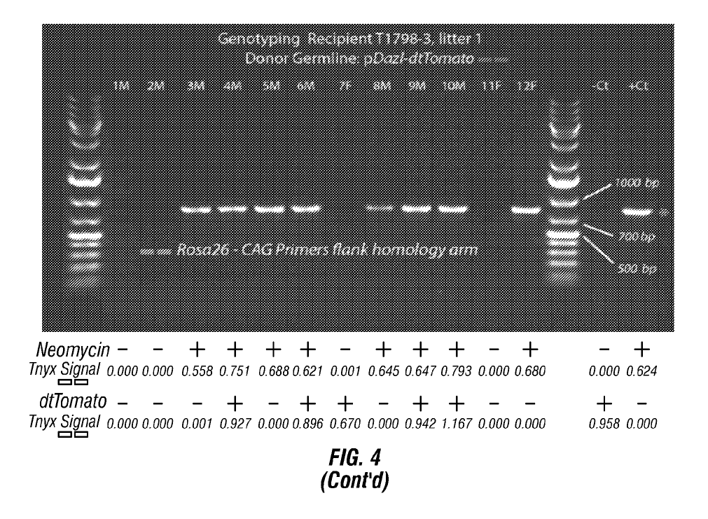

FIG. 4. Efficient Production of Inducible Cas9 Nickase Transgenic Rats by Homology Directed Repair in Donor Spermatogonial Stem Cells.

A genetically marked Brown Norway rat spermatogonial line (tgDazl-dtTomato heterozygote: USTD 2896) derived in NF9 Medium on DR4 MEFs was co-transfected with the pCAG-lox-Stop-lox-CAS9-Nickcase-Flag-PGK-Neo (tgiCas9-D10A; 20 μg) knockin construct+pX458-rRosa26a (20 μg) under conditions modified from FIGS. 2A-B, in that Neon transfected spermatogonia (˜4×10⁶ total transfected cells in 850 μl Resuspension Buffer R; 10× electroporations in 100 μl tips with 2 pulses at 1100 V, 20 m) were selected in G418-containing NF9 Medium (70 μg/ml G418) on DR4 MEFs for 6 days (d2-d8 post-transfection), and then ˜10⁵ G418 resistant spermatogonia were transplanted directly into a single testis of busulfan-treated, male sterile rats on d14 post transfection (by-passing colony picking and monoclonal expansion) (FIG. 4).

Transplanted recipient rats (n=3 rats) were paired with wild-type female Sprague Dawley rats at ˜60 days post-transplantation for breeding and the first transgenic F1 mutant progeny were produced d107 post-transplantation (FIG. 4). Based on PCR analysis using primers flanking homology arms, and sequence analyses of junction sites, ˜59% of F1 progeny (20 of 34 total pups, n=5 litters) were pure germline mutants harboring the precisely targeted 11.8 kb, tgiCas9-D10A construct in their Rosa26 locus (FIG. 4).

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Alexander et al., Proc. Nat. Acad. Sci. USA, 85:5092-5096, 1988.
Anderson, Science 256:808-813, 1992.
Barthold et al. J Urol 170, 2396-401, 2003.
Bhasin et al., J Clin Endocrinol Metab 79, 1525-9, 1994.
Bhasin, S., J Clin Endocrinol Metab 92, 1995-2004, 2007.
Bleyer et al., CA Cancer J Clin 40, 355-67, 1990.
Blomer et al., J Virol., 71(9):6641-9, 1997.
Brinster & Zimmermann, Proc Natl Acad Sci USA 91, 11298-302, 1994.
Brinster et al., Proc Natl Acad Sci USA, 91:11298-11302, 1994.
Chapman, K. M., et al. In Advanced Protocols for Animal Transgenesis: An ISTT Manual, Vol. 1, S. Pease and T. Saunders, eds. (Springer Press), pp. 237-266, 2011.
Clouthier et al., Nature, 381:418-421, 1996.
Conrad, S. et al. Nature, 2008.
Cronkhite et al., Dev. Biol., 284:171-183, 2005.
Dillon, TIBTECH 11:167-175, 1993.
Ercolani et al., J. Biol. Chem., 263:15335-15341, 1998.
European Patent Application Publication No. EP2535424 Geens, M. et al., Hum Reprod Update 14, 121-30, 2008.
Ginsburg et al., Development 110:521-528, 1990.
Haddada et al., Current Topics in Microbiology and Immunology, Doerfler and Bohm (eds), 1995.
Hamra et al., Dev Biol 2004; 269:393-410.
Hamra et al., Methods Mol Biol 2008; 450:163-179.
Hamra et al., Proc Natl Acad Sci USA 102, 17430-5, 2005.
Hamra et al., Proc Natl Acad Sci USA 99:14931-14936, 2002.
Hermann, B. P. et al. Stem Cells 25, 2330-8, 2007.
Kamtchouing et al., Biol. Reprod. 45:11-19, 1991.
Kanatsu-Shinohara et al., Biol Reprod 2008.
Kanatsu-Shinohara et al., Biol Reprod 69:612-6, 2003.
Kanatsu-Shinohara et al., Biol Reprod 69:612-616, 2003.
Kanatsu-Shinohara et al., Biol Reprod 72:985-991, 2005.
Kanatsu-Shinohara et al., Cell, 119:1001-1012, 2004.
Kanatsu-Shinohara et al., Proc Natl Acad Sci USA 103:8018-8023, 2006.
Karin et al. Cell, 36:371-379, 1989.
Kazuki et al., Gene Ther, 15:617-624, 2008.
Kossack, N. et al. “Isolation and Characterization of Pluripotent Human Spermatogonial Stem Cell-Derived Cells.” Stem Cells, 2008.
Kremer & Perricaudet, British Medical Bulletin 51(1):31-44, 1995.
Kubota et al., Proc Natl Acad Sci USA 101:16489-16494, 2004.
Macejak and Sarnow, Nature, 353:90-94, 1991.
Mahato et al., Endocrinology, 141:1273-1276, 2000.
Mahato et al., Mol Cell Endocrinol, 178:57-63, 2001.
Mann et al., Cell, 33:153-159, 1983.
Matzuk et al., Nat Med 14, 1197-213, 2008.
Michealis et al., J Vis Exp., 2014.
Miller, Nature 357:455-460, 1992.
Miller, Nature, 357:455-460, 1992.
Mitani & Caskey, TIBTECH 11:162-166, 1993.
Nabel & Feigner, TIBTECH 11:211-217, 1993.
Nagano et al., Biol Reprod, 60:1429-1436, 1999.
Nagano et al., Proc Natl Acad Sci USA 98:13090-13095, 2001.
Nagano et al., Tissue Cell 30, 389-97, 1998.
Naito, T., et al. Proc. Natl. Acad. Sci. USA 95, 5178-5181, 1998.
Naldini et al. Science 272, 263-267, 1996.
Naldini et al., 1996.
Ng, Nuc. Acid Res., 17:601-615, 1989.
Nicolas and Rubenstein, Vectors. A survey of molecular cloning vectors and their uses, Rodriguez and Denhardt (eds.), Stoneham: Butterworth, pp. 494-513, 1988.
Oates et al., Hum Reprod 17, 2813-24, 2002.
Oatley J. M. et al., Methods Enzymol. 419:259, 2006.
Ogawa et al., Nat Med, 6:29-34, 2000.
Orwig et al., Biol Reprod 67:874-879, 2002.
Paskind et al., Virology, 67:242-248, 1975.
PCT International Application Publication No. WO/2003/012143A1
PCT International Application Publication No. WO/2006/023719A2
PCT International Application Publication No. WO/2006/104370A1
PCT International Application Publication No. WO/2009/112882
PCT International Application Publication No. WO/2012/001613
Pelletier and Sonenberg, Nature, 334(6180):320-325, 1988.
Phillips et al., Phil. Trans. R. Soc. D, 365:1663-1678, 2010.
Quitsche et al., J. Biol. Chem., 264:9539-9545, 1989.
Reijo et al., Nat Genet. 10, 383-93, 1995.
Richards et al., Cell, 37:263-272, 1984.
Ryu et al., Dev Biol 274:158-170, 2004.
Ryu et al., Proc Natl Acad Sci USA 102:14302-14307, 2005.
Sadeghi-Nejad, et al., Urol J 4, 192-206, 2007.
Sambrook et al., In: Molecular cloning: a laboratory manual, 2^{nd} Ed., Cold Spring Harbor Laboratory Press, Cold Spring Harbor, N.Y., 1989.
Sander and Joung, Nature Biotechnology, 32(4):347-355, 2014.
Anderson, W. F., Science 256:808-813, 1992.
Sehgal et al. Methods Mol Biol. 1194:327-337, 2014.
Sehgal et al., Methods Mol Biol., 1194:327-337, 2014.
Shinohara et al., Proc Natl Acad Sci USA, 103:13624-13628, 2006.
Tegelenbosch R. A. and de Rooij D. G. Mutation Research 290 193-200, 1993.
Temin, Gene Transfer, Kucherlapati (ed.), New York: Plenum Press, pp. 149-188, 1986.
U.S. Pat. No. 7,732,139
U.S. Pat. No. 4,683,202
U.S. Pat. No. 4,897,355
U.S. Pat. No. 4,946,787
U.S. Pat. No. 5,049,386
U.S. Pat. No. 5,556,954
U.S. Pat. No. 5,925,565
U.S. Pat. No. 5,928,906
U.S. Pat. No. 5,935,819
U.S. Pat. No. 5,994,136
U.S. Pat. No. 5,994,136
U.S. Pat. No. 6,013,516
U.S. Pat. No. 7,627,436
U.S. Pat. No. 7,790,370
U.S. Pat. No. 8,187,811
U.S. Pat. No. 8,518,701
U.S. Patent Application Publication No. 2002/0055144
U.S. Patent Application Publication No. 2009/0148425
U.S. Patent Application Publication No. 2010/0251395
U.S. Patent Application Publication No. 2012/0222143
U.S. Patent Application Publication No. 2010/0292211
U.S. Patent Application Publication No. 2011/0039918
U.S. Patent Application Publication No. 2011/0319288
U.S. Patent Application Publication No. 2012/0136039
U.S. Patent Application Publication No. 2012/0309642
U.S. Patent Application Publication No. 2006/0204969
U.S. Patent Application Publication No. 2007/0269827
U.S. Patent Application Publication No. 2011/0104674
Van Brunt, Biotechnology 6(10):1149-1154, 1988.
Vigne, Restorative Neurology and Neuroscience 8:35-36, 1995.
Wang et al., Science, 3:343-80-4, 2014.
WO 91116024
WO 91117424
Wu et al., Cell Res. 25, 67-79, 2015.
Yu et al., Gene Therapy 1:13-26, 1994.
Zhang et al., J Cell Physiol. 211:149-158, 2007.
Zufferey et al., Nature Biotechnology, 15: 871-875, 1997.

1. A method for generating a germline modification in the genome of a mammal comprising contacting the spermatogonial stem cells (SSCs) in said mammal with Cas9 and at least one guide RNA.

2. The method of claim 1, wherein the germline modification is an insertion of a nucleic acid segment of about 10,000 to about 100,000 base pairs.

3. The method of claim 2, wherein the germline modification is an insertion of a nucleic acid segment of about 10,000 to about 50,000 base pairs, about 10,000 to about 25,000 base pairs, about 10,000 to about 20,000 base pairs, or about 10,000 to about 15,000 base pairs.

4. The method of claim 1, wherein the germline modification is an insertion of a nucleic acid segment of about 25 to about 200 base pairs.

5. The method of claim 4, wherein the germline modification is an insertion of a nucleic acid segment of about 25 to about 150 base pairs, about 25 to about 100 base pairs, about 25 to about 75 base pairs, or about 25 to about 50 base pairs.

6. The method of claim 1, wherein the SSCs are contacted with multiple guide RNAs to a plurality of target polynucleotides.

7. The method of claim 6, wherein the multiple guide RNAs include 2, 3, 4 or 5 guide RNAs.

8. The method of claim 1, wherein the Cas9 is codon optimized for expression in the SSCs.

9. The method of claim 1, wherein the germline modification comprises at least one deletion, mutation, insertion, knockout or knock-in of a gene, a gene's regulatory elements or fragment thereof.

10. The method of claim 1, wherein the germline modification comprises elimination of or a decrease in the expression of one or more gene products.

11. The method of claim 1, wherein the germline modification comprises an introduction of, or an increase in the expression of one or more gene products.

12. The method of claim 1, wherein the Cas9 and/or the at least one guide RNA are provided to the SSCs through transfection.

13. The method of claim 1, wherein the Cas9 and/or the at least one guide RNA are provided to the SSCs through electroporation.

14. The method of claim 1, wherein the method further comprises contacting the SSCs with a single-stranded oligonucleotide.

15. The method of claim 12, wherein the germline modification comprises insertion of the single-stranded oligonucleotide by non-homologous end joining (NHEJ)-mediated insertion repair or homology-directed repair (HDR).

16. A method of treating a genetic disease in a mammal caused by a disease-causing genetic mutation comprising correcting the disease-causing mutation according to the method of claim 1.

17-25. (canceled)

26. A spermatogonial stem cell comprising a germline modification obtained according to the method of claim 1.

27. A method of mating rats comprising mating of a male rat comprising the spermatogonial stem cell(s) of claim 26 with a female rat.

28-30. (canceled)

31. A stem cell medium comprising Dulbecco's Modified Eagle Medium (DMEM), Ham's F12 nutrient mixture, from about 5-7 ng/ml glial cell-derived neurotrophic factor (GDNF), from about 5-7 ng/ml Fibroblast Growth Factor-2 (FGF2), 2-mercaptoethanol, L-glutamine, and a B27 minus vitamin A supplement solution.

32-36. (canceled)

37. A stem cell composition produced by culturing a stem cell in the medium of claim 31.

38-48. (canceled)

49. A method for culturing an isolated rat spermatogonial stem cell isolated from rat testis cells comprising:
(a) allowing the isolated rat spermatogonial stem cell to adhere to a surface in a culture vessel; and
(b) culturing the rat spermatogonial stem cell in the stem cell medium comprising Dulbecco's Modified Eagle Medium (DMEM), Ham's F12 nutrient mixture, from about 5-7 ng/ml glial cell-derived neurotrophic factor (GDNF), from about 5-7 ng/ml Fibroblast Growth Factor-2 (FGF2), 2-mercaptoethanol, L-glutamine, and a B27 minus vitamin A supplement solution.

50-53. (canceled)
